(19) United States
(12) Patent Application Publication
(10) Pub. No.: US 2019/0015312 A1
(43) Pub. Date: Jan. 17, 2019

(54) COSMETIC FORMULATIONS

(71) Applicant: KIMBERLY-CLARK WORLDWIDE, INC., Neenah, WI (US)

(72) Inventors: YoenJung Lee, Yongin-si (KR); Scott W. Wenzel, Neenah, WI (US); Stacy A. Mundschau, Weyauwega, WI (US)

(21) Appl. No.: 16/066,258

(22) PCT Filed: Dec. 29, 2015
(86) PCT No.: PCT/US15/67856

§ 371 Date: Jun. 26, 2018

(51) Int. Cl. A61K 8/64; A61K 8/26; A61K 8/06; A61K 8/04; A61K 8/73; A61K 8/37; A61Q 19/08

(52) U.S. Cl. CPC A61K 8/64; A61K 8/26; A61K 8/06; A61K 2800/48; A61K 8/731; A61K 8/375; A61Q 19/08; A61K 8/042

(57) ABSTRACT

A cosmetic formulation can include a salt based cosmetic astringent and an active peptide. The salt based cosmetic astringent can form at least about 0.1% by total weight of the cosmetic formulation. The active peptide can include at least one of (i) a dipeptide including Arginine and Tyrosine and (ii) an oligopeptide with at least four amino acids.

### TECHNICAL FIELD

The present invention relates to cosmetic formulations and to a method for combining a high level of salt based cosmetic astringents with peptides while not inactivating the peptides.

### BACKGROUND OF THE DISCLOSURE

Certain salts provide a potent astringent or pore tightening effect, which for many types of cosmetics provides an aesthetic benefit and lends credence to the products efficacy while providing a short-term drying effect. For products that contain active ingredients that require a longer duration to achieve clinical benefits, salt based cosmetic astringents can provide an immediate, perceptible change in how the skin feels, promoting the use of the product for a long enough period to ensure the active ingredient can achieve its desired effect. One such set of active ingredients include peptides, which are limited in the ability to be combined with salt based cosmetic astringents, particularly highly water soluble aluminum salts which, when bound to the peptide cause conformation changes that render it inactive.

It is well known to those of ordinary skill in the art to avoid using compounds with a high ionic strength within formulations containing peptides. The process referred to as “salting out” is a common method to separate proteins from solution. Within the published literature, there are several documents which discuss the effect that high electrolyte concentrations have on peptides. See, e.g., The Effect of Concentrated Salt Solutions on the Activity Coefficient of Acetyltetraglycine Ethyl Ester (Dwight R. Robinson, William P. Jencks, J. Am. Chem. Soc., 1965, 87 (11), pp 2470-2479), Effects of salts on the free energy of the peptide group(Pradip K. Nandi, Dwight R. Robinson, J. Am. Chem. Soc., 1972, 94 (4), pp 1299-1308), and Effects of Adsorption to Aluminum Salt Adjuvants on the Structure and Stability of Model Protein Antigens (LaToya S. Jones, Laura J. Peek, Jonathan Power, Aaron Markham, Brian Yazzie and C. Russell Middaugh, Apr. 8, 2005 The Journal of Biological Chemistry, 280, 13406-13414).

Thus, what is needed are cosmetic formulations including peptides which demonstrate activity in the presence of salt based cosmetic astringents to provide consumers both an immediate, sensory benefit and drying effect that will promote product usage long enough to ensure that the desired physiological effects take place. These effects may include, but are not limited to, up regulation of collagen or elastin or inhibiting release or otherwise affecting the uptake of acetylcholine or other neurotransmitters of interest. These effects then provide treatments targeted at the signs of aging, hyperhidrosis, or the strength of the skin. Additionally, it would also be beneficial to provide such cosmetic formulations in the form of an emulsion, which in some aspects, could be sprayable to provide increased convenience for the user.

### SUMMARY OF THE DISCLOSURE

In one embodiment, a cosmetic formulation can include a salt based cosmetic astringent and an active peptide. The salt based cosmetic astringent can provide at least about 0.1% by total weight of the cosmetic formulation. The active peptide can include at least one of (i) a dipeptide including Arginine and Tyrosine and (ii) an oligopeptide with at least four amino acids.

In another embodiment, a cosmetic formulation can include a salt based cosmetic astringent selected from the group consisting of: Ammonium and Potassium Alum, Aluminum Triphosphate, Aluminum Glycinate and Aluminum Phenolsulfate, Alcloxa, Aldioxa, Aluminum Stearate, Aluminum Sulfate and Aluminum Citrate, Sodium Aluminum Phosphate, Sodium Alum, Sodium Aluminum Chlorohydroxy Lactate, Calcium Lactate, Calcium Chloride, Calcium Sulfate Hydrate, Sodium Aluminum Lactate, Zinc Acetate, Zinc Chloride, Zinc Sulfate, Zinc Lactate, Zinc Zeolite, Zinc Phenolsulfonate, and combinations thereof. The cosmetic formulation can also include an active peptide including at least one of (i) a dipeptide including Arginine and Tyrosine and (ii) an oligopeptide with at least four amino acids. The salt based cosmetic astringent can provide at least about 0.1% by total weight of the cosmetic formulation.

In yet another embodiment, a cosmetic formulation can include a salt based cosmetic astringent and an active peptide. The salt based cosmetic astringent can provide at least about 0.1% by total weight of the cosmetic formulation. The active peptide can include at least one of (i) Acetyl Dipeptide-1 Cetyl Ester and (ii) an Acetyl Hexapeptide-20 combined with Pentapeptide-18.

### DETAILED DESCRIPTION OF THE DISLOSURE

The cosmetic formulations on the present disclosure provide a formulation including a peptide that unexpectedly remains active in the presence of salt based cosmetic astringents. Such cosmetic formulations can provide consumers both an immediate, sensory benefit and drying effect from the salt based cosmetic astringent as well longer term desired physiological effects including regulation of collagen or elastin or inhibiting release or otherwise affecting the uptake of acetylcholine or other neurotransmitters of interest. As noted above, these effects can provide treatments targeted at the signs of aging, hyperhidrosis, or the strength of the skin.

In some aspects, the cosmetic formulations of the present disclosure can be in the form of an emulsion. For the preferred embodiments of emulsions, all of the formulations of the present disclosure display a stability that has not heretofore been observed. The emulsions do not break over time and are resistant to breaking down under conditions of temperature extremes, particularly elevated temperature. Furthermore, the preferred emulsions of the present disclosure display stability under conditions of repeated heating and cooling cycles. It is unexpected that the emulsions of the present disclosure display stability over time as well as over temperature elevation and variability. As used herein, a physically stable emulsion is defined as one that has a consistent appearance and no oil/water phase separation for one month at 50° C., and three months at 40° C. However, the process by which the emulsions are made can affect long-term physical stability. The process of the present disclosure includes making the emulsion before adding salt based cosmetic astringents, such as Aluminum salts, thereto. Adding salt based cosmetic astringents to the composition before or during emulsion formation can result in an unstable emulsion.

In one aspect of the disclosure, the formulations can be sprayable. The term “sprayable” refers to the ability to spray the formulations with a hand-pump spray bottle, a hand-squeeze spray bottle, pressurized aerosol cans, or similar devices. For purposes of this disclosure, “sprayable” formulations are those that are able to dispense through the Calmar Mark VI® dispenser commercially produced by Mead Westvaco Corporation. The specifications of the spray head of this dispenser are a 20 mm cap with 410 thread, an overall spray volume of 0.16 cc, a spray diameter of 0.057 inches and a dip tube of 2.75 inches. The formulation was loaded into a 2 oz. Boston Round bottle available from Poly-Tainer Inc. (20/410 thread). If the formulation was able to disperse from the package within 10 pumps, the formulation is deemed as “sprayable.” Other than the pressure applied from the manual depression of the pump, no other pressure is present within the packaging (i.e. aerosolized, pressurized CO2, etc). In some embodiments, preferred formulations produce a v-shaped pattern of the formulation upon spraying that give droplets upon the skin. Of course, it is contemplated that other conventional spray dispenser mechanism can be used to dispense the skin protectant formulation, including, but not limited to, aerosol or pressurized propellant dispensers, motor driven pump dispensers, and other dispensers using manual spray pump mechanisms. In some embodiments, an emulsion can be sprayable and have a viscosity of about 1 to about 15,000 cps, or about 1 to about 10,000 cps, or about 500 to 8,000 cps.

In one embodiment, the formulation of the present disclosure may be utilized with a continuous spray dispenser. Continuous spray, or continuously sprayable, technology is meant to indicate that the formulation provides any-angle spraying and uniform coverage. An example of a continuous spray dispenser would include a flexible, expandable container adapted to receive the skin protectant formulation. The flexible container is removable surrounded by a rigid exterior housing or canister, which is provided with an air-tight seal. The canister is sealed prior to filling the flexible container with the formulation, so that air is trapped within the canister in the volume unoccupied by the flexible container. When the flexible container is filled with the formulation, the container expands, thereby compressing the air within the canister. While maintaining complete separation from the formulation, this compressed air acts as a propellant. The compressed air then acts against the flexible container to uniformly propel the formulation from the container. In this example, there is no need to pump the spray like conventional spray dispensers to distribute the formulation onto skin. This is advantageous in limiting pain for those with limited dexterity or arthritis.

In another exemplary continuous spray dispenser, the container may include a pump that is integral with the cap on the dispenser. In this example, the air is compressed in the canister not when sealing the canister, but by pumping air into the canister to provide compressed air as a propellant. The compressed air added by a consumer then acts against the flexible container to uniformly propel the skin protectant formulation out of the container.

Continuous spray technology is well known in the art. Suitable commercially available continuous spray dispensers for use with the skin protectant formulation can include, for example, the 12HS Dry Spray Dispenser commercially available from Rexam Airspray or the bag-on-valve dispenser commercially available from ColepCCL.

### Peptides

Peptides of the present disclosure that keep efficacy in their activity in the presence of salt based cosmetic astringents are referred to herein as “active peptides.” Active peptides demonstrating preferable results, as will be discussed further herein, include a dipeptide including Arginine and Tyrosine and oligopeptides that include at least four amino acids. Examples of oligopeptides that include at least four amino acids are Inyline (Acetyl Hexapeptide-30) alone or in combination with Leuphasyl (Pentapeptide-18), Pentapeptide-3 (Vialox), Palmitoyl Hexapeptide-19 (BoNT-L) and Palmitoyl heptapeptide-18 (X50 Myocept—which can be provided at 0.001% active peptide solution). An example of a dipeptide that includes Arginine and Tyrosine is Acetyl Dipeptide-1 Cetyl Ester (Calmosensine).

Particularly preferred active peptides include combination of Inyline (Acetyl Hexapeptide-30) and Leuphasyl (Pentapeptide-18) as well as Acetyl Dipeptide-1 Cetyl Ester (Calmosensine), Palmitoyl Hexapeptide-19 (BoNT-L).

Peptides can be provided in solid form or in solution form. When in solution form, concentrations of these peptides generally do not exceed 5.0% w/w individually as supplied by the manufacturer in solution form, and in some circumstances can have much lower concentrations, such as 0.0005% w/w, and thus, use of peptides in solutions may utilize more peptide solution as compared to peptides provided in solid form to provide the same weight percentage of the active peptides in the resultant formulation. As will be discussed in further detail below and supported by examples, in some embodiments, it is preferable to have cosmetic formulations of the present disclosure have an active peptide that comprises between about 0.0001% to about 1.0000% by total weight of the cosmetic formulation, taking into consideration the dilution of the active peptides in solution form. For example, if a 5% w/w peptide solution is added to the formulation at 5% w/w by weight of the formulation, then the active peptide will form 0.25% by total weight of the formulation. In more preferable embodiments, the active peptide can comprise about 0.0002% to about 0.5000% by total weight of the cosmetic formulation.

### Salt Based Cosmetic Astringents

As used herein, a salt based cosmetic astringent is defined as salt based cosmetic ingredients intended to induce a tightening or tingling sensation on the skin. Exemplary salt based cosmetic astringents include Ammonium and Potassium Alum, Aluminum Triphosphate, Aluminum Glycinate and Aluminum Phenolsulfate, Alcloxa, Aldioxa, Aluminum Stearate, Aluminum Sulfate and Aluminum Citrate, Sodium Aluminum Phosphate, Sodium Alum, Sodium Aluminum Chlorohydroxy Lactate, Calcium Lactate, Calcium Chloride, Calcium Sulfate Hydrate, Sodium Aluminum Lactate, Zinc Acetate, Zinc Chloride, Zinc Sulfate, Zinc Lactate, Zinc Zeolite, Zinc Phenolsulfonate, and combinations thereof. A salt based cosmetic astringent does not include any anti-perspirant based compounds which are listed on the United States Food and Drug Administration's Anti-Perspirant Monograph (21 C.F.R. §§ 310, 350, and 369. Federal Register Vol. 68, No. 110). Anti-perspirant based compounds listed on the Anti-Perspirant Monograph, and which are not “salt based cosmetic astringents as used herein, include: (a) Aluminum chloride up to 15 percent, calculated on the hexahydrate form, in an aqueous solution nonaerosol dosage form; (b) Aluminum chlorohydrate up to 25 percent; (c) Aluminum chlorohydrex polyethylene glycol up to 25 percent; (d) Aluminum chlorohydrex propylene glycol up to 25 percent; (e) Aluminum dichlorohydrate up to 25 percent; (f) Aluminum dichlorohydrex polyethylene glycol up to 25 percent; (g) Aluminum dichlorohydrex propylene glycol up to 25 percent; (h) Aluminum sesquichlorohydrate up to 25 percent; (i) Aluminum sesquichlorohydrex polyethylene glycol up to 25 percent; (j) Aluminum sesquichlorohydrex propylene glycol up to 25 percent; (k) Aluminum zirconium octachlorohydrate up to 20 percent; (l) Aluminum zirconium octachlorohydrex gly up to 20 percent; (m) Aluminum zirconium pentachlorohydrate up to 20 percent; (n) Aluminum zirconium pentachlorohydrex gly up to 20 percent; (o) Aluminum zirconium tetrachlorohydrate up to 20 percent; (p) Aluminum zirconium tetrachlorohydrex gly up to 20 percent; (q) Aluminum zirconium trichlorohydrate up to 20 percent; and (r) Aluminum zirconium trichlorohydrex gly up to 20 percent.

Particularly preferred salt based cosmetic astringents include Ammonium Alum, Potassium Alum, Aluminum Triphosphate, Aluminum Glycinate, Aluminum Phenolsulfate, Alcloxa, Aldioxa, Aluminum Stearate, Aluminum Sulfate, Aluminum Citrate, Sodium Aluminum Phosphate, Sodium Alum, Sodium Aluminum Chlorhydroxy Lactate, Sodium Aluminum Lactate, and any combinations thereof. Especially preferred salt based cosmetic astringents include the Aluminum salt based cosmetic astringents, and particularly, Ammonium Alum, Aldioxa, Aluminum Stearate, and Aluminum Citrate.

Salt based cosmetic astringents can be supplied in solid form or in solutions. One advantage of using salt based cosmetic astringents to other astringents is the dry powdery feel they impart to the formulation and ability to keep the surface of the skin dry, particularly aluminum salt based cosmetic astringents. In some embodiments, a salt based cosmetic astringent can provide at least about 0.1% by total weight of the cosmetic formulation of the present disclosure. Preferably, a salt based cosmetic astringent can provide between about 0.1% to about 15% by total weight of the formulation, more preferably between about 0.1% to about 10%, and even more preferably about 0.1% to about 8% by total weight of the formulation.

It is further understood that the cosmetic formulations of the present disclosure can include sufficient levels of the positive ion of the salt (e.g., Aluminum, Calcium, Zinc) of the salt based cosmetic astringent, which can provide the immediate sensory effect that is desired. The formulations of the present disclosure therefore can include a minimum of 0.01% by weight of the formulation of the positive ion of the salt based cosmetic astringent as calculated on a molar basis. In some embodiments, the formulations of the present disclosure can include between about 0.01% to about 5.00% by weight of the formulation of the positive ion as calculated on a molar basis. More preferably, the formulations of the present disclosure can include between about 0.025% to about 4.00%, or about 0.05% to about 3.00%, or about 0.075% to about 2.00%, or about 0.10% to about 1.00% by weight of the formulation of the positive ion as calculated on a molar basis. These ranges of weights calculated on a molar basis for the positive ion of the salt based cosmetic astringent are particularly relevant to the Aluminum salt based cosmetic astringents.

To calculate the percent of the positive ion of the salt based cosmetic astringent in a cosmetic formulation on a molar basis, the molecular weight of the entire molecule of the salt based cosmetic astringent is first calculated. The molecular weight of the positive ion is then divided by the entire molecular weight of the compound and multiplied by 100 to arrive at the weight percentage of the positive ion in the salt based cosmetic astringent. Then the weight percentage of the positive ion in the salt based cosmetic astringent is multiplied by the weight percentage of the salt based cosmetic astringent in the overall cosmetic formulation and further multiplied by 100 to provide the weight percentage of the positive ion in the cosmetic formulation on a molar basis. For example, the molecular weight of Aluminum Citrate is 216 g/mol. The Aluminum contributes 26.98 g/mol, and taking 26.98 g/mol divided by 216 g/mol provides a molar weight percentage of 12.49% of Aluminum for Aluminum Citrate. Thus, for “Batch 4” in Table A described below (where the Aluminum Citrate provides 8% of the weight percentage of the formulation), the Aluminum can provide about 1.0% of the molar weight of the cosmetic formulation (i.e., 0.1249×0.08×100=0.9992%). For one of ordinary skill in the art, this molar calculation can be easily completed for other positive ions of the salt based cosmetic astringents in the cosmetic formulations of the present disclosure.

Not only can the salt based cosmetic astringents affect the activity of a peptide, but it is believed that salt based cosmetic astringents can also significantly disrupt the stability of emulsions. Not to be bound by theory, but it is believed that this instability is due to the high ionic strength of the salt based cosmetic astringents. The formulations of the present disclosure provided in the form of an emulsion can include a relatively high level of one or more salt based cosmetic astringents, yet still remain stable.

### Emulsifiers:

Suitable emulsifiers that can produce a stable emulsion with water phase thickeners include: a combination of EMALEX 602 (Steareth-2)/EMALEX 620 (Steareth-20) and combination of EMALEX 602/EMALEX 620/CUTINA GMS (Glyceryl Stearate). In addition to emulsifiers, surfactants such as fatty alcohols Cetyl alcohol and/or Stearyl alcohol can be used as viscosity increasing agents.

It is noted that not all emulsifiers will produce a stable emulsion with water-based thickeners. The following emulsifiers, used either alone or in combination, failed to produce a physically stable emulsion when combined with or without the thickeners outlined below and 5% Aluminum based Salt: ARLACEL 165 (Glyceryl stearate, PEG-100 stearate), CERALUTION H (Behenyl alcohol, Glyceryl stearate, Glyceryl stearate citrate, Disodium ethylene dicocamide PEG-15 disulfate), DERMOFEEL EASYMULS (Sunflower seed acids, Polyglyceryl-3 esters citrate, Helianthus annuus (sunflower) seed oil), EMALEX 840 (PEG-40 stearate), EMALEX HC-60 (PEG-60 hydrogenated castor oil), EMALEX SEG-07 (Glyceryl stearate, PEG-100 stearate), EMULGADE PL 68/50 (Cetearyl glucoside, Cetearyl alcohol), EMULGADE SUCRO (Sucrose polystearate, Hydrogenated polyisobutene), EUMULGIN SG (Sodium Stearoyl Glutamate), EUMULGIN SML 20 (Polysorbate 20), INCROQUAT TMS-50 (Behentrimonium Methosulfate, Cetyl Alcohol, Butylene Glycol), and TWEEN 60 (Polysorbate 60).

### Thickeners:

Thickeners affect the viscosity of the emulsion and help prevent oil droplets from coalescing, leading to emulsion instability. As used herein, a thickener can also be referred to as a rheology modifier. Suitable water phase thickeners include: ARAGUM 3173 (Xanthan gum, Guar gum, Propylene glycol alginate), AVICEL PC 611 (Microcrystalline cellulose, Cellulose gum), BENTONE GEL CAO V (Ricinus communis, Stearalkonium hectorite, Propylene carbonate), KRUCEL HPC (Hydroxypropyl cellulose), NATPURE Gum (Gum Arabic) and NATROSOL HEC (Hydroxyethyl cellulose). It is noted that gums such as Xanthan Gum, Guar Gum, Gum Arabic, etc. tended to thicken with the addition of the salt based cosmetic astringent of Aluminum Sulfate Hydrate. Celluloses or minerals used alone precipitated with the addition of the salt based cosmetic astringent of Aluminum Sulfate Hydrate. However, a combination of Gums and Celluloses/Minerals demonstrated better emulsion stability and desirable viscosities. Where thicker formulations are preferred the usage of Sepigel 305 (Polyacrylamide (and) C13-14 Isoparaffin (and) Laureth-7) is preferred given its ability to thicken in high electrolyte formulations.

### Method:

In one aspect, the present disclosure includes a method for making a cosmetic formulation in the form of a stable emulsion. Shown in TABLE A are the base formulations which contain combinations of salt based cosmetic astringents of aluminum salts (Batch 3 and 4), a base formulation including 6.25% of salt based OTC monograph antiperspirant of active Aluminum Chlorohydrate to serve as a control (Batch 2), as well as base formulations containing no aluminum salts (Batch 1). These sample emulsions were prepared using the following method.

Referring to Table A, a water phase is created by adding water soluble PART A ingredients (Methylparaben, Chlorphenisin, Microcrystalline Cellulose, Aluminum Citrate, Ammonium Alum and Sodium Hydroxide) to water as it is heated to a temperature of 75 degrees Centigrade (° C.). An oil phase is created by blending the PART B ingredients: Sunflower oil, Steareth-2, Steareth-21, Glyceryl Stearate and Aluminum Stearate. Oil phase ingredients are mixed constantly while they are being heated to 75° C. Once both phases reach 75° C., Part B is added to Part A and homogenized at 5000 rpm for 5 minutes using a Silverson Homogenizer. The emulsion is returned to propeller based mixing until it cools to 35° C. or lower at which point, the ingredients of Part C were added. The pH of the formulation is then adjusted to 5.25 or 6.75 using a sodium hydroxide and/or malic acid. Within some preparations it may be advantageous to add the water soluble aluminum salts after the emulsion cools to 35° C. and prior to pH adjusting. In all cases, the peptide of interest (as will be discussed further below) was added last to these base formulations to ensure that both the temperature and pH of the batch was appropriate to ensure stability.

TABLE A

| | | Batch 1 | Batch 2 | Batch 3 | Batch 4 |
|---|---|---|---|---|---|
| Trade Name | INCI Name | Wt (%) | Wt (%) | Wt (%) | Wt (%) |
| Part A | | | | | |
| Water | Water | 88.79 | 74 | 72 | 73 |
| COSEPT M | Methylparaben | 0.2 | 0.3 | 0.3 | 0.3 |
| ELSTAB CPN | Chlorphenesin | 0.1 | 0.2 | 0.2 | 0.2 |
| RHODACARE XC | Xanthan Gum | 0.3 | 0.0 | 0.0 | 0.0 |
| AVICEL 611 | Microcrystalline | 0.0 | 1 | 1 | 1 |
| | Cellulose and | | | | |
| | Cellulose Gum | | | | |
| Part B | | | | | |
| RITA SSO | Sunflower Oil | 4.0 | 4 | 4 | 4 |
| BRIJ-2 | Steareth-2 | 2.6 | 1.5 | 1.5 | 1.5 |
| BRIJ-21 | Steareth-21 | 0.6 | 3 | 3 | 3 |
| CUTINA GMS | Glyceryl Stearate | 0 | 2 | 2 | 2 |
| Aluminum Distearate | Aluminum Distearate | 0 | 0 | 1 | 0 |
| Part C | | | | | |
| REACH 501 solution | Aluminum | 0 | 12.5 | 0 | 0 |
| (50% active) | Chlorohydrate | | | | |
| Aluminum Citrate | Aluminum Citrate | 0 | 0 | 1.5 | 8 |
| Ammonium Alum | Ammonium Alum | 0 | 0 | 5 | 0 |
| Sodium Hydroxide (20%) | Sodium Hydroxide | 0 | QA | QA | QA |

### Optional Ingredients:

(a) pH Adjusting Agent

The cosmetic formulations of the present disclosure may further include a pH-adjusting agent. Such agents are desirable for the creation of cosmetic formulations having a pH at or near that of human skin. Therefore, the pH can typically be adjusted as necessary so that the cosmetic formulations of the present disclosure can have a pH of from 4 to 7, or more desirably, from 4.5 to 6.5. The pH can be adjusted by adding one or more pH-adjusting agents in an amount effective to provide such pH values (“effective amount”). Agents that may be used to adjust the pH of the cosmetic formulations include organic and inorganic acids and bases.

Acid pH-adjusting agents include organic acids which are relatively non-irritating. Such acids include malic acid, citric acid acetic acid, propionic acid, oxalic acid, glycolic acid, malonic acid, lactic acid, succinic acid, tartaric acid, aspartic acid, maleic acid, glutaric acid, glutamic acid, gluconic acid, sorbic acid, benzoic acid, ascorbic acid, salicylic acid and mixtures thereof. In one aspect of the present disclosure, a desirable pH-adjusting agent is malic acid.

The amount of the pH-adjusting agent that is employed depends on the equivalent weight of the pH-adjusting agent and the desired pH. Typically, the pH-adjusting agent is used in an amount of from about 0.05% to about 0.5% by weight of the cosmetic formulations. Desirable cosmetic formulations of the present disclosure include from about 0.1% to about 0.5% percent, and typically about 0.2% to about 0.3% of the pH-adjusting agent by weight of the cosmetic formulation.

(b) Preservatives

The cosmetic formulations of the present disclosure may further include one or more preservatives. Preservatives function in one or more ways to improve the shelf life of the cosmetic formulations and products incorporating same. For example, the preservative may be an anti-microbial agent, an anti-bacterial agent, an anti-fungal agent, or a combination thereof.

Preservatives herein include, but are not limited to, benzethonium chloride, benzisothiazolinone, benzoic acid, benzyl alcohol, 2-Bromo-2-nitropropane-1,3-diol, butylparaben, caprylyl glycol, chlorhexidine digluconate, DMDM hydantoin, diazolidinyl urea, dehydroacetic acid, ethylparaben, iodopropynyl butylcarbamate, methylchloroisothiazolinone, methylisothiazolinone, methyldibromo glutaronitrile, methylparaben, pentylene glycol, phenethyl alcohol, phenoxyethanol, propylparaben, polyaminopropyl biguanide, quaternium-15, salicylic acid, sodium benzoate, sodium methylparaben, sodium dehydroacetate, thymol, triclosan and mixtures thereof.

In one aspect of the disclosure, benzoic acid, with or without phenoxyethanol, is effective in preventing the growth of a wide variety of microbes and fungi.

An anti-microbial agent may be used in an amount that is effective to provide desired shelf life (storage stability, i.e., microorganisms do not grow to a significant extent) (herein alternatively referred to as “an effective amount”). This includes demonstrating sufficient anti-microbial activity in accordance with United States Pharmacopeia test entitled “Microbial Test, Antimicrobial Preservative—Effectiveness”.

(c) Chelating Agent

The cosmetic formulations may contain one or more chelating agents. The chelating agent tends to bind metals (e.g., calcium ions, magnesium ions) that may be present in the cosmetic formulation so as to enhance the efficiency of the emulsifier and the anti-microbial agent. Thus, the chelating agent may be considered to provide a level of anti-microbial activity to function as a preservative. The chelating agent may be used in an amount that is effective to bind the aforementioned metals (hereinafter alternatively referred to as an “effective amount”), typically an amount ranging from about 0.01% to about 0.2% by weight of the emulsion. Particularly preferred cosmetic formulations include from about 0.05% to about 0.2% by weight of the cosmetic formulation, more preferably from about 0.05% to about 0.10% by weight of the cosmetic formulation. Chelating agents and their use in personal cleansing emulsions are well known in the art. Exemplary chelating agents include disodium EDTA, trisodium EDTA, tetrasodium EDTA, and tetrasodium iminodisuccinate.

(d) Emollients

In one embodiment, the cosmetic formulations can optionally include one or more emollients, which typically act to soften, soothe, and otherwise lubricate and/or moisturize the skin. Suitable emollients that can be incorporated into the compositions include oils such as alkyl dimethicones, alkyl methicones, alkyldimethicone copolyols, phenyl silicones, alkyl trimethylsilanes, dimethicone, dimethicone crosspolymers, cyclomethicone, lanolin and its derivatives, fatty esters, glycerol esters and derivatives, propylene glycol esters and derivatives, alkoxylated carboxylic acids, alkoxylated alcohols, fatty alcohols, and combinations thereof.

The cosmetic formulations may include one or more emollients in an amount of from about 0.01% (by total weight of the cosmetic formulation) to about 20% (by total weight of the cosmetic formulation), or from about 0.05% (by total weight of the cosmetic formulation) to about 10% (by total weight of the cosmetic formulation), or from about 0.10% (by total weight of the cosmetic formulation) to about 5% (by total weight of the cosmetic formulation).

(e) Additional Astringents

Optionally, the cosmetic formulations of the present disclosure may also include other, non-salt based ingredients which provide an astringent effect to the skin. The majority of these compounds are generally recognized as cosmetic astringents and can be used between a concentration of 0.1-10% as supplied in combination with the salt based cosmetic astringent, such as aluminum salt. It is generally believed that the tannin content of these extracts is responsible for the pore tightening effect demonstrated. Non-limiting examples of these materials include: Hamamelis Virginiana (Witch Hazel) Bark/Leaf/Twig Extract, Mentha Viridis (Spearmint) Leaf Oil, Ribes Nigrum (Black Currant) Fruit, Ribes Rubrum (Currant) Fruit, Cinnamomum Camphora (Camphor) Gum Extract, Citrus Aurantifolia (Lime) Flower Extract, Citrus Aurantium Bergamia (Bergamot) Leaf Oil, Citrus Limon (Lemon) Fruit Oil, Ginkgo Biloba Nut Extract, Hamamelis Virginiana (Witch Hazel) Bark/Twig Extract, Tannic Acid, Hydrolyzed Walnut Extract, Gynostemma Pentaphyllum Leaf Extract, Alchemilla Vulgaris Leaf Extract, Ethanol and Gallic Acid.

(f) Other

The cosmetic formulations of the present disclosure may optionally include other ingredients, e.g., fragrance; skin soothing aids such as aloe, lavender, chamomile, green tea, calendula, etc.; skin moisturizers (humectants) such as glycerin, propylene glycol, betaine, and hydroxyethyl urea; or emollients other than those previously described; powders and the like.

In one aspect, a fragrance is added in a concentration range of 0.05% to 4.0% by total weight of the cosmetic formulation. In another aspect, the fragrance is added in a concentration of 1.20% by total weight of the cosmetic formulation.

### Applications:

The formulation may be a simple solution, a serum, or an emulsion, with the latter being the most preferred product form. The emulsion may be applied to the skin as a spray or a cream. As discussed above, the spray embodiments can be sprayed by pump spray, squeeze spray, and pressurized aerosols, among other options.

The cosmetic formulations can also be dispensed in a wipe. The wipe may have a cellulosic structure, such as a tissue, a non-woven structure, foam or a combination thereof that has a one-ply or that has a multi-ply structure. Suitable wipe substrates include conventional nonwoven materials, homogeneous paper, through-air-dried paper, a differential-density paper, or a differential-basis weight paper or foam.

### EXPERIMENTS AND EXAMPLES

The effectiveness of the peptide material at addressing perspiration was evaluated in a human clinical study. The screening of various potential sweat-reducing materials was determined using at least 20 volunteer human subjects. Twenty-four subjects were planned for enrollment into the study to ensure that 20 subjects completed testing. Baseline screening was performed on up to 40 subjects to ensure a sufficient number of subjects were enrolled in the treatment application phase who met minimum sweat production criteria of 50 mg over a 20-minute collection period. As shown in Table C and Table D, each subject had seven test materials randomly applied to the lower back in 2 inch by 2 inch treatment application areas and one site was left untreated on each subject as a control. The study subjects participated in a 4-day test period consisting of four, supervised treatment applications, spaced 24-hours apart, and one post treatment application sweat collection period 1-hour post-product application on Day 4. The study staff visually assessed the application sites, and applied each of the test materials to the backs of the subjects. The study proceeded as follows:
Day 0, Baseline screening performed and eligibility for testing assessed.
Day 1, subjects had the test materials applied and the sites occluded for 24-hours.
Day 2, patches removed, test sites wiped, test site visually assessed, second product application with sites being occluded.
Day 3, patches removed, test sites wiped, test site visually assessed, third product application with sites being occluded.
Day 4, patches removed, test sites wiped, test site visually assessed, fourth product application with sites being occluded for 1-hour prior to removal and sweat collection period (60-minutes in a room maintained at 100° F.±2° F. and 35% humidity±5%).
   The gravimetric measurements of perspiration following removal of the final patches was performed according to the following procedures:
Subjects entered a temperature controlled room maintained at 100° F.±2° F. and 35% humidity±5%. Subjects were instructed to not leave the room during the approximately 60-minute test period.
Absorbent gauze sponges, 2 inch by 2 inch (two 4-ply sponges, Avant Gauze® Sponges), were applied to the eight sites on the lower back of the subjects. Sponges were occluded on the site using 3M™ Blenderm™ Medical Tape, and affixed to the site using Scanpor® Paper Tape.
The sponges remained in place for a 20-minute acclimation period. Water was available in the temperature controlled room for subjects to consume during the acclimation period, and between patch/sponge applications.
Following the acclimation period, the sponges were removed and discarded.
A set of pre-weighed sponges (pre-weight includes affixed tape) was applied to each test site.
The pre-weighed sponges remained in place for a 20-minute period. Subjects sat in a chair during the 20-minute period with both feet flat on the ground, with arms at their sides. Subjects were not allowed to consume water during this 20-minute period.
Following the 20-minute period, the sponges with the 3M™ Blenderm™ Medical Tape were removed and sealed in a bag or container for weighing within 1-hour of removal.
A second set of pre-weighed sponges was applied to each test site. The sponges with the 3M™ Blenderm™ Medical Tape were weighed prior to application.
The second set of pre-weighed sponges remained in place for a 20-minute period. Subjects sat in a chair during the 20-minute period with both feet flat on the ground, with arms at their sides. Subjects were not allowed to consume water during this 20-minute period.
Following the 20-minute period, the sponges with the 3M™ Blenderm™ Medical Tape were removed and sealed in a bag or container for weighing within 1-hour of removal.
After exiting the temperature-controlled room, the subjects again sat in normal ambient indoor temperature for at least 20-minutes, and trained technicians determined the body temperature, pulse, and blood pressure of each subject. Water was available for subjects to consume during this period.
If the vital signs were not within the normal range, the subjects remained in normal ambient indoor temperature for additional 20-minute periods, and the vitals rechecked.
Subjects failing to return to normal vital sign ranges were considered as having an adverse event(s), and referred for medical treatment.
Each set of sponges with medical tape was weighed, and the weight subtracted from the pre-application weight to determine the amount of perspiration produced. The weight of perspiration from the two patches was summed, and used in the statistical analysis.

Table B provides the formulations that include peptides where the base composition listed is that in Table A. In all instances, water was removed on a 1:1 basis to include the ingredient listed. These cosmetic formulations were prepared in a manner consistent with the methodology outlined in the Method section above.

TABLE B

| | | | | Wt (%) of |
|---|---|---|---|---|
| | | Trade Name of Peptide | | Peptide |
| Formulation | Base | Solution Used | Peptide Name | Solution |
| A | 1 | Vialox (>90% active | Pentapeptide-3 | 0.3 |
| | | peptide) | | |
| B | 1 | Inyline (0.05% active | Acetyl Hexapeptide-30 (additional | 5 |
| | | peptide) | ingredients - Water, Arginine, | |
| | | | Caprylyl Glycol) | |
| C | 1 | Inyline (0.05% active | Acetyl Hexapeptide-30 (additional | 5 |
| | | peptide) | ingredients - Water, Arginine, | |
| | | | Caprylyl Glycol) | |
| | | Leuphasyl (0.05% | Pentapeptide-18 (additional | 5 |
| | | active peptide) | ingredients - Water, Glycerin, | |
| | | | Caprylyl Glycol) | |
| D | 1 | Calmosensine (1.0% | Acetyl Dipeptide-1 Cetyl Ester | 3 |
| | | active peptide) | (additional ingredients - Butylene | |
| | | | Glycol, Water, Laureth-3, | |
| | | | Hydroxyethylcellulose) | |
| E | 1 | BoNT-L (0.004-0.006% | Palmitoyl Hexapeptide-19 (additional | 5 |
| | | active peptide) | ingredients - Water, Sodium | |
| | | | Levulinate, Sodium Anisate) | |
| F | 1 | Syn-AKE (0.1-1.0% | Dipeptide Diaminobutyroyl | 0.25 |
| | | active peptide) | Benzylamide Diacetate (additional | |
| | | | ingredients - Water, Glycerin) | |
| G | 2 | None | None | 0 |
| H | 3 | None | None | 0 |
| I | 3 | Calmosensine (1.0% | Acetyl Dipeptide-1 Cetyl Ester | 3 |
| | | active peptide) | (additional ingredients - Butylene | |
| | | | Glycol, Water, Laureth-3, | |
| | | | Hydroxyethylcellulose) | |
| J | 3 | Inyline (0.05% active | Acetyl Hexapeptide-30 (additional | 5 |
| | | peptide) | ingredients - Water, Arginine, | |
| | | | Caprylyl Glycol) | |
| | | Leuphasyl (0.05% | Pentapeptide-18 (additional | 5 |
| | | active peptide) | ingredients - Water, Glycerin, | |
| | | | Caprylyl Glycol) | |
| K | 3 | BoNT-L (0.004-0.006% | Palmitoyl Hexapeptide-19 (additional | 5 |
| | | active peptide) | ingredients - Water, Sodium | |
| | | | Levulinate, Sodium Anisate) | |
| L | 4 | None | None | 0 |
| M | 4 | Calmosensine (1.0% | Acetyl Dipeptide-1 Cetyl Ester | 3 |
| | | active peptide) | (additional ingredients - Butylene | |
| | | | Glycol, Water, Laureth-3, | |
| | | | Hydroxyethylcellulose) | |

### Experimental Data:

Table C contains the anti-perspirant test results for the formulations listed in Table B which contain the base composition 1 (from Table A). Table D contains the anti-perspirant test results for the formulations list in Table B which contain the base compositions 3 and 4 (from Table A). Those cosmetic formulations including base composition 1 were tested in separate clinical evaluation than the cosmetic formulations including base compositions 3 and 4. Table E provides the stability results for base composition 2 as well as formulations G-J (which include base compositions 2 and 3) demonstrating that the cosmetic formulations were stable.

TABLE C

| | | | | | | | | | Control |
|---|---|---|---|---|---|---|---|---|---|
| | | G | A | B | C | D | E | F | Site |
| % Change in | n | 23 | 22 | 23 | 22 | 23 | 23 | 23 | 23 |
| Perspiration | | | | | | | | | |
| | Mean | −44.1 | −24.6 | −17.6 | −27.8 | −24.7 | −26.9 | −17.6 | 11.48 |
| | (SD) | (30.7) | (33.4) | (37.9) | (40.9) | (43.9) | (33.9) | (40.1) | (67.7) |
| | Median | −45.1 | −21 | −20.1 | −37 | −36.2 | −41 | −25.6 | 2.19 |
| Signed | p-value | <.0001 | 0.0083 | 0.0618 | 0.0022 | 0.0066 | 0.0014 | 0.0066 | |
| Rank Test | | | | | | | | | |
| Compared | | | | | | | | | |
| Against | | | | | | | | | |
| Control | | | | | | | | | |
| Signed | p-value | | 0.0034 | <.0001 | 0.0201 | 0.0024 | 0.001 | <.0001 | |
| Rank Test | | | | | | | | | |
| Compared | | | | | | | | | |
| Against #G | | | | | | | | | |

Reduced amount of perspiration = Test-Day 4 perspiration - Pre-screen perspiration.
† Sum denotes Baseline and Test-Day 4 perspiration as sum of collection A and B.
Signed Rank Test is performed on amount of perspiration for pairwise comparison between test materials.

As shown in Table C, the cosmetic formulations including the combination of Leuphasyl and Inyline (C), Calmosensine (D) and BoNT-L (E) provided sweat reduction levels similar to that of a 6.25% Aluminum Chlorohydrate formulation (12.5% solution of 50% active) (G).

TABLE D

| | | | | | | | | | Control |
|---|---|---|---|---|---|---|---|---|---|
| | | G | H | I | J | K | L | M | Site |
| % Change in | n | 32 | 32 | 32 | 32 | 32 | 32 | 32 | 32 |
| Perspiration | | | | | | | | | |
| | Mean | −48.0 | −25.7 | −44.2 | −46.6 | −33.2 | −30.3 | −52.6 | −22.6 |
| | (SD) | (73.5) | (114) | (51.7) | (46.5) | (64.2) | (59.7) | (40.7) | (61.3) |
| | Median | −66.8 | −48.9 | −62.1 | −62 | −52.4 | −46.7 | −55.7 | −38.7 |
| Signed Rank | p-value | <.0001 | 0.1223 | 0.0017 | 0.0067 | 0.132 | 0.0719 | 0.0008 | |
| Test | | | | | | | | | |
| Compared | | | | | | | | | |
| Against | | | | | | | | | |
| Control Site | | | | | | | | | |
| Signed Rank | p-value | | 0.0659 | 0.2685 | 0.1642 | 0.0159 | 0.0021 | 0.3983 | |
| Test | | | | | | | | | |
| Compared #2 | | | | | | | | | |

Reduced amount of perspiration = Test-Day4 perspiration - Pre-screen perspiration.
† Sum denotes Baseline and Test-Day4 perspiration as sum of collection A and B.
Signed Rank Test is performed on amount of perspiration for pairwise comparison between test materials.

As shown in Table D, the cosmetic formulations including the combination of Leuphasyl and Inyline (J) or Calmosensine (l) provided sweat reduction similar to that of the 6.25% Aluminum Chlorohydrate formulation (12.5% solution of 50% active) (G) despite the presence of the aluminum salts in formulations (J) and (l). This result demonstrates that the peptides selected for formulations (J) and (l) maintained the activity demonstrated in Table C (as comparable to formulations (C) and (D)) in the presence of the salt based cosmetic astringent, which is an unexpected result. Additionally, formulation (M) including Calmosensine provided sweat reduction despite the presence of a salt based cosmetic astringent, maintaining the activity demonstrated in Table C (as comparable to formulation (D)). The cosmetic formulation (K) including BoNT-L provided sweat reduction as well despite the presence of the salt based cosmetic astringent. Formulation (K) can be compared to formulation (E) in Table C, which shows that the peptide of formulation (K) provided activity in the presence of a salt based cosmetic astringent to a lesser extent than formulations (J) and (l). Those codes with salt based cosmetic astringents alone (H and L) demonstrated no significant change in efficacy when compared to the Aluminum Chlorohydrate formulation (G), an OTC monograph anti-perspirant that served as a control.

Thus, the cosmetic formulations including a peptide of either (i) a dipeptide including Arginine and Tyrosine or (ii) an oligopeptide with at least four amino acids unexpectedly provided high activity in the presence of a salt based cosmetic astringent. Specifically, the dipeptide of Acetyl Dipeptide-1 Cetyl Ester showed high activity within the presence of a salt based cosmetic astringent. Additionally, the oligopeptides of Acetyl Hexapeptide-30 and Pentapeptide-18 also showed high activity within the presence of a salt based cosmetic astringent. It is believed that other dipeptides including Arginine and Tyrosine and other oligopeptides with at least four amino acids would provide similar levels of activity in the presence of salt based cosmetic astringents.

Table E demonstrates that the emulsion remained stable for a minimum of 3 months at 40° C. and through three freeze-thaw cycles.

TABLE E

| | | | | | 3 Month 40 C. | | 4 Month 40 C. | |
|---|---|---|---|---|---|---|---|---|
| | Room Temp | | F/T | | Samples | | Samples | |
| Formulation | pH | Viscosity | pH | Viscosity | Viscosity | pH | pH | Viscosity |
| 2 | | | 5.31 | 17920 | 3840 | 5.26 | 7467 | 5.06 |
| G | 6.78 | 7925 | 6.78 | 8440 | 9600 | 6.75 | NA | NA |
| H | 6.75 | 6890 | 6.75 | 9325 | 9624 | 6.46 | NA | NA |
| I | 6.77 | 7900 | 6.77 | 10250 | 7720 | 6.79 | NA | NA |
| J | 6.03 | 12900 | 6.03 | 16400 | 17200 | 5.89 | NA | NA |

Note:
Only data related to a stable result are reported.

### Embodiments

Embodiment 1: A cosmetic formulation comprising: a salt based cosmetic astringent, the salt based cosmetic astringent comprising at least about 0.1% by total weight of the cosmetic formulation; and an active peptide including at least one of (i) a dipeptide including Arginine and Tyrosine and (ii) an oligopeptide with at least four amino acids.
Embodiment 2: The cosmetic formulation of embodiment 1, wherein the salt based cosmetic astringent comprises between about 0.1% to about 15.0% by total weight of the cosmetic formulation.

Embodiment 3: The cosmetic formulation of embodiment 1 or embodiment 2, wherein the active peptide is a dipeptide including Arginine and Tyrosine.
Embodiment 4: The cosmetic formulation of embodiment 3, wherein the active peptide includes Acetyl Dipeptide-1 Cetyl Ester.
Embodiment 5: The cosmetic formulation of embodiment 1 or embodiment 2, wherein the active peptide is an oligopeptide with at least four amino acids.
Embodiment 6: The cosmetic formulation of embodiment 5, wherein the active peptide is selected from the group consisting of: Acetyl Hexapeptide-20, Pentapeptide-18, Acetly Dipeprtide-1 Cetyl Ester, Pentapeptide-3, Palmitoyl Hexapeptide-19, and Palmitoyl Heptapeptide-18, and any combinations thereof.
Embodiment 7: The cosmetic formulation of any one of the preceding embodiments, wherein the active peptide comprises between about 0.0001% to about 1.0000% by total weight of the cosmetic formulation.
Embodiment 8: The cosmetic formulation of any one of the preceding embodiments, wherein the salt based cosmetic astringent is selected from the group consisting of: Ammonium and Potassium Alum, Aluminum Triphosphate, Aluminum Glycinate and Aluminum Phenolsulfate, Alcloxa, Aldioxa, Aluminum Stearate, Aluminum Sulfate and Aluminum Citrate, Sodium Aluminum Phosphate, Sodium Alum, Sodium Aluminum Chlorohydroxy Lactate, Calcium Lactate, Calcium Chloride, Calcium Sulfate Hydrate, Sodium Aluminum Lactate, Zinc Acetate, Zinc Chloride, Zinc Sulfate, Zinc Lactate, Zinc Zeolite, Zinc Phenolsulfonate, and combinations thereof.
Embodiment 9: The cosmetic formulation of any one of embodiments 1-7, wherein the salt based cosmetic astringent comprises aluminum.
Embodiment 10: The cosmetic formulation of any one of the preceding embodiments, wherein the cosmetic formulation is in the form of an emulsion, gel, or serum.
Embodiment 11: The cosmetic formulation of embodiment 10, wherein the cosmetic formulation is in the form of an emulsion including a water phase and an oil phase; wherein the water phase comprises water and a rheology modifier selected from the group consisting of: Microcrystalline Cellulose, Cellulose Gum, and combinations thereof; and wherein the oil phase comprises Steareth-2, Glyceryl Stearate, and an emulsifier selected from the group consisting of: Steareth-20, Steareth-21, and combinations thereof.
Embodiment 12: A cosmetic formulation comprising: a salt based cosmetic astringent selected from the group consisting of: Ammonium and Potassium Alum, Aluminum Triphosphate, Aluminum Glycinate and Aluminum Phenolsulfate, Alcloxa, Aldioxa, Aluminum Stearate, Aluminum Sulfate and Aluminum Citrate, Sodium Aluminum Phosphate, Sodium Alum, Sodium Aluminum Chlorohydroxy Lactate, Calcium Lactate, Calcium Chloride, Calcium Sulfate Hydrate, Sodium Aluminum Lactate, Zinc Acetate, Zinc Chloride, Zinc Sulfate, Zinc Lactate, Zinc Zeolite, Zinc Phenolsulfonate, and combinations thereof; and an active peptide including at least one of (i) a dipeptide including Arginine and Tyrosine and (ii) an oligopeptide with at least four amino acids; the salt based cosmetic astringent comprising at least about 0.1% by total weight of the cosmetic formulation.
Embodiment 13: The cosmetic formulation of embodiment 12, wherein the salt based cosmetic astringent comprises between about 0.1% to about 15.0% by total weight of the cosmetic formulation.
Embodiment 14: The cosmetic formulation of embodiment 12 or embodiment 13, wherein the active peptide comprises between about 0.0001% to about 1.0000% by total weight of the cosmetic formulation.
Embodiment 15: The cosmetic formulation of any one of embodiments 12-14, wherein the cosmetic formulation is in the form of an emulsion including a water phase and an oil phase; wherein the water phase comprises water and a rheology modifier selected from the group consisting of: Microcrystalline Cellulose, Cellulose Gum, and combinations thereof; and wherein the oil phase comprises Steareth-2, Glyceryl Stearate, and an emulsifier selected from the group consisting of: Steareth-20, Steareth-21, and combinations thereof.
Embodiment 16: A cosmetic formulation comprising: a salt based cosmetic astringent, the salt based cosmetic astringent comprising at least about 0.1% by total weight of the cosmetic formulation; and an active peptide including at least one of (i) Acetyl Dipeptide-1 Cetyl Ester and (ii) an Acetyl Hexapeptide-20 combined with Pentapeptide-18.
Embodiment 17: The cosmetic formulation of embodiment 16, wherein the salt based cosmetic astringent is selected from the group consisting of: Ammonium and Potassium Alum, Aluminum Triphosphate, Aluminum Glycinate and Aluminum Phenolsulfate, Alcloxa, Aldioxa, Aluminum Stearate, Aluminum Sulfate and Aluminum Citrate, Sodium Aluminum Phosphate, Sodium Alum, Sodium Aluminum Chlorohydroxy Lactate, Calcium Lactate, Calcium Chloride, Calcium Sulfate Hydrate, Sodium Aluminum Lactate, Zinc Acetate, Zinc Chloride, Zinc Sulfate, Zinc Lactate, Zinc Zeolite, Zinc Phenolsulfonate, and combinations thereof.
Embodiment 18: The cosmetic formulation of embodiment 16 or embodiment 17, wherein the active peptide comprises between about 0.0001% to about 1.0000% by total weight of the cosmetic formulation.
Embodiment 19: The cosmetic formulation of any one of embodiments 16-18, wherein the salt based cosmetic astringent comprises between about 0.1% to about 15.0% by total weight of the cosmetic formulation.
Embodiment 20: The cosmetic formulation of any one of embodiments 16-19, wherein the cosmetic formulation is in the form of an emulsion including a water phase and an oil phase; wherein the water phase comprises water and a rheology modifier selected from the group consisting of: Microcrystalline Cellulose, Cellulose Gum, and combinations thereof; and wherein the oil phase comprises Steareth-2, Glyceryl Stearate, and an emulsifier selected from the group consisting of: Steareth-20, Steareth-21, and combinations thereof.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

1. A cosmetic formulation comprising:
a salt based cosmetic astringent, the salt based cosmetic astringent comprising at least about 0.1% by total weight of the cosmetic formulation; and
an active peptide including at least one of (i) a dipeptide including Arginine and Tyrosine and (ii) an oligopeptide with at least four amino acids.

2. The cosmetic formulation of claim 1, wherein the salt based cosmetic astringent comprises between about 0.1% to about 15.0% by total weight of the cosmetic formulation.

3. The cosmetic formulation of claim 1, wherein the active peptide is a dipeptide including Arginine and Tyrosine.

4. The cosmetic formulation of claim 3, wherein the active peptide includes Acetyl Dipeptide-1 Cetyl Ester.

5. The cosmetic formulation of claim 1, wherein the active peptide is an oligopeptide with at least four amino acids.

6. The cosmetic formulation of claim 5, wherein the active peptide is selected from the group consisting of: Acetyl Hexapeptide-20, Pentapeptide-18, Acetly Dipeprtide-1 Cetyl Ester, Pentapeptide-3, Palmitoyl Hexapeptide-19, and Palmitoyl Heptapeptide-18, and any combinations thereof.

7. The cosmetic formulation of claim 1, wherein the active peptide comprises between about 0.0001% to about 1.0000% by total weight of the cosmetic formulation.

8. The cosmetic formulation of claim 1, wherein the salt based cosmetic astringent is selected from the group consisting of: Ammonium and Potassium Alum, Aluminum Triphosphate, Aluminum Glycinate and Aluminum Phenolsulfate, Alcloxa, Aldioxa, Aluminum Stearate, Aluminum Sulfate and Aluminum Citrate, Sodium Aluminum Phosphate, Sodium Alum, Sodium Aluminum Chlorohydroxy Lactate, Calcium Lactate, Calcium Chloride, Calcium Sulfate Hydrate, Sodium Aluminum Lactate, Zinc Acetate, Zinc Chloride, Zinc Sulfate, Zinc Lactate, Zinc Zeolite, Zinc Phenolsulfonate, and combinations thereof.

9. The cosmetic formulation of claim 1, wherein the salt based cosmetic astringent comprises aluminum.

10. The cosmetic formulation of claim 1, wherein the cosmetic formulation is in the form of an emulsion, gel, or serum.

11. The cosmetic formulation of claim 1, wherein the cosmetic formulation is in the form of an emulsion including a water phase and an oil phase; wherein the water phase comprises water and a rheology modifier selected from the group consisting of: Microcrystalline Cellulose, Cellulose Gum, and combinations thereof; and wherein the oil phase comprises Steareth-2, Glyceryl Stearate, and an emulsifier selected from the group consisting of: Steareth-20, Steareth-21, and combinations thereof.

12. A cosmetic formulation comprising:
a salt based cosmetic astringent selected from the group consisting of: Ammonium and Potassium Alum, Aluminum Triphosphate, Aluminum Glycinate and Aluminum Phenolsulfate, Alcloxa, Aldioxa, Aluminum Stearate, Aluminum Sulfate and Aluminum Citrate, Sodium Aluminum Phosphate, Sodium Alum, Sodium Aluminum Chlorohydroxy Lactate, Calcium Lactate, Calcium Chloride, Calcium Sulfate Hydrate, Sodium Aluminum Lactate, Zinc Acetate, Zinc Chloride, Zinc Sulfate, Zinc Lactate, Zinc Zeolite, Zinc Phenolsulfonate, and combinations thereof; and
an active peptide including at least one of (i) a dipeptide including Arginine and Tyrosine and (ii) an oligopeptide with at least four amino acids;
the salt based cosmetic astringent comprising at least about 0.1% by total weight of the cosmetic formulation.

13. The cosmetic formulation of claim 12, wherein the salt based cosmetic astringent comprises between about 0.1% to about 15.0% by total weight of the cosmetic formulation.

14. The cosmetic formulation of claim 12, wherein the active peptide comprises between about 0.0001% to about 1.0000% by total weight of the cosmetic formulation.

15. The cosmetic formulation of claim 12, wherein the cosmetic formulation is in the form of an emulsion including a water phase and an oil phase; wherein the water phase comprises water and a rheology modifier selected from the group consisting of: Microcrystalline Cellulose, Cellulose Gum, and combinations thereof; and wherein the oil phase comprises Steareth-2, Glyceryl Stearate, and an emulsifier selected from the group consisting of: Steareth-20, Steareth-21, and combinations thereof.

16. A cosmetic formulation comprising:
a salt based cosmetic astringent, the salt based cosmetic astringent comprising at least about 0.1% by total weight of the cosmetic formulation; and
an active peptide including at least one of (i) Acetyl Dipeptide-1 Cetyl Ester and (ii) an Acetyl Hexapeptide-20 combined with Pentapeptide-18.

17. The cosmetic formulation of claim 16, wherein the salt based cosmetic astringent is selected from the group consisting of: Ammonium and Potassium Alum, Aluminum Triphosphate, Aluminum Glycinate and Aluminum Phenolsulfate, Alcloxa, Aldioxa, Aluminum Stearate, Aluminum Sulfate and Aluminum Citrate, Sodium Aluminum Phosphate, Sodium Alum, Sodium Aluminum Chlorohydroxy Lactate, Calcium Lactate, Calcium Chloride, Calcium Sulfate Hydrate, Sodium Aluminum Lactate, Zinc Acetate, Zinc Chloride, Zinc Sulfate, Zinc Lactate, Zinc Zeolite, Zinc Phenolsulfonate, and combinations thereof.

18. The cosmetic formulation of claim 16, wherein the active peptide comprises between about 0.0001% to about 1.0000% by total weight of the cosmetic formulation.

19. The cosmetic formulation of claim 16, wherein the salt based cosmetic astringent comprises between about 0.1% to about 15.0% by total weight of the cosmetic formulation.

20. The cosmetic formulation of claim 16, wherein the cosmetic formulation is in the form of an emulsion including a water phase and an oil phase; wherein the water phase comprises water and a rheology modifier selected from the group consisting of: Microcrystalline Cellulose, Cellulose Gum, and combinations thereof; and wherein the oil phase comprises Steareth-2, Glyceryl Stearate, and an emulsifier selected from the group consisting of: Steareth-20, Steareth-21, and combinations thereof.
